(19) United States
(12) Patent Application Publication
(10) Pub. No.: US 2010/0016692 A1
(43) Pub. Date: Jan. 21, 2010

(54) SYSTEMS AND METHODS FOR COMPUTING A PHYSIOLOGICAL PARAMETER USING CONTINUOUS WAVELET TRANSFORMS

(75) Inventors: Paul Stanley Addison, Edinburgh (GB); James Nicholas Watson, Dunfermline (GB)

Correspondence Address: Nellcor Puritan Bennett LLC;ATTN: IP Legal, 6135 Gunbarrel Avenue, Boulder, CO, 80301, US

(73) Assignee: Nellcor Puritan Bennett Ireland, Galway (IE)

(21) Appl. No.: 12/245,451

(22) Filed: Oct. 3, 2008

Related U.S. Application Data
(60) Provisional application No. 61/081,019, filed on Jul. 15, 2008.

(51) Int. Cl. A61B 5/08; A61B 5/1455; A61B 5/02; A61B 5/021; A61B 5/024

(52) U.S. Cl. 600/324; 600/508; 600/485; 600/529

(57) ABSTRACT

According to embodiments, systems and methods for computing a physiological parameter are provided. The physiological parameter may be calculated using a continuous wavelet transform technique as well as using a non-continuous wavelet transform technique. More than one value for the physiological parameter may be calculated using various techniques. The values may be evaluated to select a desired value, or an average or weighted average of the values may be computed to generate a desired value.

### CROSS REFERENCE TO RELATED APPLICATION

This application claims the benefit of U.S. Provisional Application No. 61/081,019, filed Jul. 15, 2008 which is hereby incorporated by reference herein in its entirety.

### SUMMARY

The present disclosure relates to signal processing and, more particularly, the present disclosure relates to computing a physiological parameter using one or more continuous wavelet transform techniques and non-continuous wavelet transform techniques. Physiological parameters may be computed using a physiological signal generated via a patient monitoring device such an oximeter or other sensor device. An oximeter, for example, may be used to measure physiological parameters associated with blood flow, such as oxygen saturation. Many different types of techniques may be used to calculate a physiological parameter Each technique may generate a different value based on the same physiological signal. As will be described further herein, such values obtained using different calculation techniques may be analyzed to select a desired value or values. Since information indicating a physiological parameter may be used to assess a patient's condition, it is important that the information about the physiological parameter be as accurate and reliable as possible.

Although the present disclosure refers to PPG signals for illustrative purposes, the present disclosure is applicable to any suitable signals. Those skilled in the art will recognize that the present disclosure has wide applicability to other signals including, but not limited to other biosignals (e.g., electrocardiogram, electroencephalogram, electrogastrogram, electromyogram, heart rate signals, pathological sounds, ultrasound, or any other suitable biosignal), dynamic signals, non-destructive testing signals, condition monitoring signals, fluid signals, geophysical signals, astronomical signals, electrical signals, financial signals including financial indices, sound and speech signals, chemical signals, meteorological signals including climate signals, and/or any other suitable signal, and/or any combination thereof.

In one example, a physiological measurement system may take a pulse oximetry signal from a patient and then analyze the pulse oximetry signal to measure, derive, or compute one or more physiological parameters. These physiological parameters may include, for example, pulse rate, respiration rate, oxygen saturation, blood pressure, respiration effort, or other parameters.

The physiological parameters may be calculated using a variety of techniques, some of which may be based on use of a Continuous Wavelet Transform (CWT) technique. An estimate or calculation of such physiological parameters may also be achieved using other non-CWT techniques, such as a spectral or time domain technique.

Each CWT and non-CWT technique may have an advantage or disadvantage in its use for calculating a physiological parameter. The CWT and non-CWT techniques may also be used in combination to provide additional advantages, for example, in efficiency, accuracy or reliability. For example SpO₂ (i.e., arterial blood oxygen saturation) may be calculated quickly using a ratio of ratios technique and CWT techniques (e.g., using wavelet ratio or Lissajous techniques) with the two results combined, for example, by taking an average to obtain an improved value for the calculated saturation. Alternatively, oxygen saturation may be calculated using both the ratio of ratios technique and CWT techniques. One of the results may be selected that is closest to an expected (e.g., historical) value to obtain a desired value for the calculated oxygen saturation. Information gained by one of the techniques may also be used by the second for improved accuracy. For example, the pulse rate obtained by a time domain method may be used by a CWT method (e.g., ridge tracking) to obtain a more accurate or continuous value for pulse rate or other physiological parameters. A comparison of multiple calculations obtained using different techniques, such as CWT, non-CWT, and combination techniques, may be performed to obtain a desired value. Such a desired value may be based on certain advantages and disadvantages of techniques, a comparison of the calculations against standards or thresholds, or other data.

An embodiment is provided for a method, system, and computer readable medium including instructions, for determining a physiological parameter from a physiological signal. The physiological signal is received. A first value for a physiological parameter may be calculated based at least in part on the at least one physiological signal using a continuous wavelet transform technique. A second value for the physiological parameter may be calculated based at least in part on the at least one physiological signal using a non-continuous wavelet transform technique. The two values are then analyzed to determine a desired value for the physiological parameter. Analysis of the two values may include considering: an expected range of values for the physiological parameter, historical information, patient information, a statistical measure, noise associated with the signal, a confidence indicator, and any other suitable data. The desired value may be an average of the two values or a weighted average of the two values based on weights assigned to each of the values. The weights may be based on, for example, an expected range of values for the physiological parameter, historical information, patient information, a statistical measure, noise associated with the signal, a confidence indicator, any other suitable data, or any suitable combination thereof. Some of the physiological parameters calculated herein may include blood oxygen saturation, pulse rate, respiration rate, blood pressure, and respiration effort. A continuous wavelet transform technique may includes performing a continuous wavelet transform of the at least one physiological signal, generating at least one scalogram based at least in part on the continuous wavelet transform, and analyzing features in the at least one scalogram. Analyzing features of the scalogram may include techniques such as ridge following, generating a Lissajous figure based on amplitude values of two scalograms, and determining a ratio of an amplitude value of one scalogram to an amplitude value of another scalogram. Some non-continuous wavelet transform techniques may include time domain techniques and a spectral technique.

### BRIEF DESCRIPTION OF THE DRAWINGS

The above and other features of the present disclosure, its nature and various advantages will be more apparent upon consideration of the following detailed description, taken in conjunction with the accompanying drawings in which:

FIG. 1 shows an illustrative pulse oximetry system in accordance with an embodiment;

FIG. 2 is a block diagram of the illustrative pulse oximetry system of FIG. 1 coupled to a patient in accordance with an embodiment;

FIGS. 3(a) and 3(b) show illustrative views of a scalogram derived from a PPG signal in accordance with an embodiment;

FIG. 3(c) shows an illustrative scalogram derived from a signal containing two pertinent components in accordance with an embodiment;

FIG. 3(d) shows an illustrative schematic of signals associated with a ridge in FIG. 3(c) and illustrative schematics of a further wavelet decomposition of these newly derived signals in accordance with an embodiment;

FIGS. 3(e) and 3(f) are flow charts of illustrative steps involved in performing an inverse continuous wavelet transform in accordance with embodiments;

FIG. 4 is a block diagram of an illustrative continuous wavelet processing system in accordance with some embodiments;

FIG. 5 shows an illustrative method for computing a physiological parameter in accordance with an embodiment; and

FIG. 6 shows an illustrative method for computing a physiological parameter in accordance with another embodiment.

### DETAILED DESCRIPTION

An oximeter is a medical device that may determine the oxygen saturation of the blood. One common type of oximeter is a pulse oximeter, which may indirectly measure the oxygen saturation of a patient's blood (as opposed to measuring oxygen saturation directly by analyzing a blood sample taken from the patient) and changes in blood volume in the skin. Ancillary to the blood oxygen saturation measurement, pulse oximeters may also be used to measure the pulse rate of the patient. Pulse oximeters typically measure and display various blood flow characteristics including, but not limited to, the oxygen saturation of hemoglobin in arterial blood.

An oximeter may include a light sensor that is placed at a site on a patient, typically a fingertip, toe, forehead or earlobe, or in the case of a neonate, across a foot. The oximeter may pass light using a light source through blood perfused tissue and photoelectrically sense the absorption of light in the tissue. For example, the oximeter may measure the intensity of light that is received at the light sensor as a function of time. A signal representing light intensity versus time or a mathematical manipulation of this signal (e.g., a scaled version thereof, a log taken thereof, a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate the amount of the blood constituent (e.g., oxyhemoglobin) being measured as well as the pulse rate and when each individual pulse occurs.

The light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. Red and infrared wavelengths may be used because it has been observed that highly oxygenated blood will absorb relatively less red light and more infrared light than blood with a lower oxygen saturation. By comparing the intensities of two wavelengths at different points in the pulse cycle, it is possible to estimate the blood oxygen saturation of hemoglobin in arterial blood.

When the measured blood parameter is the oxygen saturation of hemoglobin, a convenient starting point assumes a saturation calculation based on Lambert-Beer's law. The following notation will be used herein:

I(λ,t)=Iₒ(λ)exp(−(sβₒ(λ)+(1−s)βᵣ(λ))l(t)) (1)

where:
λ=wavelength;
t=time;
I=intensity of light detected;
Iₒ=intensity of light transmitted;
s=oxygen saturation;
βₒ, βᵣ=empirically derived absorption coefficients; and
l(t)=a combination of concentration and path length from emitter to detector as a function of time.

The traditional approach measures light absorption at two wavelengths (e.g., red and infrared (IR)), and then calculates saturation by solving for the “ratio of ratios” as follows.
1. First, the natural logarithm of (1) is taken (“log” will be used to represent the natural logarithm) for IR and Red

log I=log Iₒ−(sβₒ+(1−s)βᵣ)l (2)
2. (2) is then differentiated with respect to time

$$\frac{d log I}{d t} = - ( s {β}_{o} + ( 1 - s ) {β}_{r} ) \frac{d l}{d t} \qquad ( 3 )$$

3. Red (3) is divided by IR (3)

$$\frac{d log I ( {λ}_{R} ) / d t}{d log I ( {λ}_{IR} ) / d t} = \frac{s {β}_{o} ( {λ}_{R} ) + ( 1 - s ) {β}_{r} ( {λ}_{R} )}{s {β}_{o} ( {λ}_{IR} ) + ( 1 - s ) {β}_{r} ( {λ}_{IR} )} \qquad ( 4 )$$

4. Solving for s

$$s = \frac{\frac{d log I ( {λ}_{IR} )}{d t} {β}_{r} ( {λ}_{R} ) - \frac{d log I ( {λ}_{R} )}{d t} {β}_{r} ( {λ}_{IR} )}{\begin{matrix}\frac{d log I ( {λ}_{R} )}{d t} ( {β}_{o} ( {λ}_{IR} ) - {β}_{r} ( {λ}_{IR} ) ) - \\ \frac{d log I ( {λ}_{IR} )}{d t} ( {β}_{o} ( {λ}_{R} ) - {β}_{r} ( {λ}_{R} ) )\end{matrix}}$$

Note in discrete time

$$\frac{d log I ( λ , t )}{d t} ≃ log I ( λ , {t}_{2} ) - log I ( λ , {t}_{1} )$$

### Using log A-log B=log A/B,

$$\frac{d log I ( λ , t )}{d t} ≃ log ( \frac{I ( {t}_{2} , λ )}{I ( {t}_{1} , λ )} )$$

So, (4) can be rewritten as

$$\frac{\frac{d log I ( {λ}_{R} )}{d t}}{\frac{d log I ( {λ}_{IR} )}{d t}} ≃ \frac{log ( \frac{I ( {t}_{1} , {λ}_{R} )}{I ( {t}_{2} , {λ}_{R} )} )}{log ( \frac{I ( {t}_{1 ,} {λ}_{IR} )}{I ( {t}_{2} , {λ}_{IR} )} )} = R \qquad ( 5 )$$

where R represents the “ratio of ratios.” Solving (4) for s using (5) gives

$$s = \frac{{β}_{r} ( {λ}_{R} ) - R {β}_{r} ( {λ}_{IR} )}{R ( {β}_{o} ( {λ}_{IR} ) - {β}_{r} ( {λ}_{IR} ) ) - {β}_{o} ( {λ}_{R} ) + {β}_{r} ( {λ}_{R} )} .$$

From (5), R can be calculated using two points (e.g., PPG maximum and minimum), or a family of points. One method using a family of points uses a modified version of (5). Using the relationship

$$\frac{d log I}{d t} = \frac{d I / d t}{I} \qquad ( 6 )$$

now (5) becomes

$$\begin{matrix}\frac{\frac{d log I ( {λ}_{R} )}{d t}}{\frac{d log I ( {λ}_{IR} )}{d t}} ≃ \frac{\frac{I ( {t}_{2} , {λ}_{R} ) - I ( {t}_{1} , {λ}_{R} )}{I ( {t}_{1} , {λ}_{R} )}}{\frac{I ( {t}_{2} , {λ}_{IR} ) - I ( {t}_{1} , {λ}_{IR} )}{I ( {t}_{1} , {λ}_{IR} )}} \\ = \frac{[ I ( {t}_{2} , {λ}_{R} ) - I ( {t}_{1} , {λ}_{R} ) ] I ( {t}_{1} , {λ}_{IR} )}{[ I ( {t}_{2} , {λ}_{IR} ) - I ( {t}_{1} , {λ}_{IR} ) ] I ( {t}_{1} , {λ}_{R} )} \\ = R\end{matrix} \qquad ( 7 )$$

which defines a cluster of points whose slope of y versus x will give R where

x(t)=[I(t₂,λ_{IR})−I(t₁,λ_{IR})]I(t₁,λ_{R})

y(t)=[I(t₂,λ_{R})−I(t₁,λ_{R})]I(t,λ_{IR})

y(t)=Rx(t) (8)

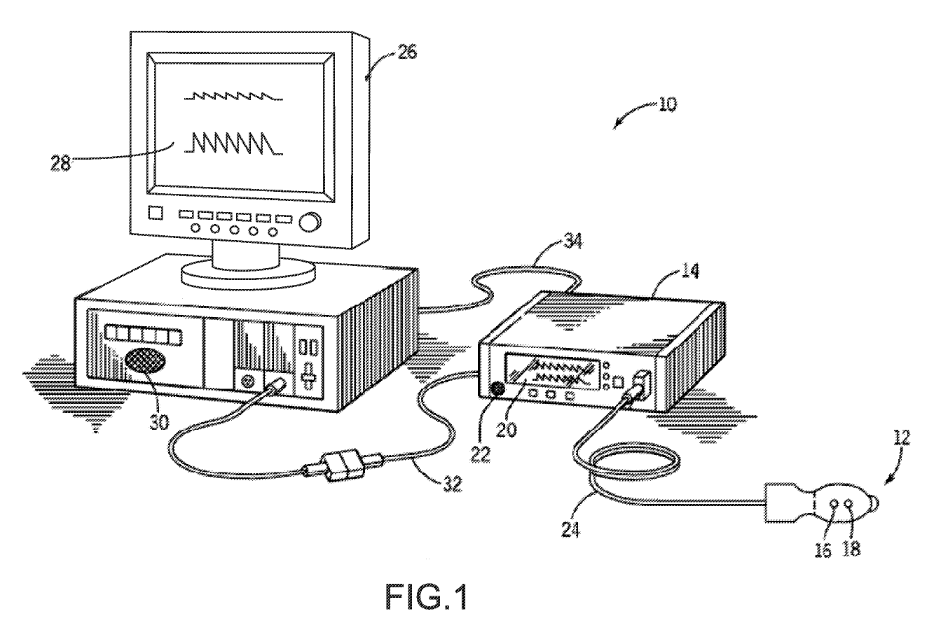

FIG. 1 is a perspective view of an embodiment of a pulse oximetry system 10. System 10 may include a sensor 12 and a pulse oximetry monitor 14. Sensor 12 may include an emitter 16 for emitting light at two or more wavelengths into a patient's tissue. A detector 18 may also be provided in sensor 12 for detecting the light originally from emitter 16 that emanates from the patient's tissue after passing through the tissue.

According to an embodiment, system 10 may include a plurality of sensors forming a sensor array in lieu of single sensor 12. Each of the sensors of the sensor array may be a complementary metal oxide semiconductor (CMOS) sensor. Alternatively, each sensor of the array may be charged coupled device (CCD) sensor. In another embodiment, the sensor array may be made up of a combination of CMOS and CCD sensors. The CCD sensor may comprise a photoactive region and a transmission region for receiving and transmitting data whereas the CMOS sensor may be made up of an integrated circuit having an array of pixel sensors. Each pixel may have a photodetector and an active amplifier.

According to an embodiment, emitter 16 and detector 18 may be on opposite sides of a digit such as a finger or toe, in which case the light that is emanating from the tissue has passed completely through the digit. In an embodiment, emitter 16 and detector 18 may be arranged so that light from emitter 16 penetrates the tissue and is reflected by the tissue into detector 18, such as a sensor designed to obtain pulse oximetry data from a patient's forehead.

In an embodiment, the sensor or sensor array may be connected to and draw its power from monitor 14 as shown. In another embodiment, the sensor may be wirelessly connected to monitor 14 and include its own battery or similar power supply (not shown). Monitor 14 may be configured to calculate physiological parameters based at least in part on data received from sensor 12 relating to light emission and detection. In an alternative embodiment, the calculations may be performed on the monitoring device itself and the result of the oximetry reading may be passed to monitor 14. Further, monitor 14 may include a display 20 configured to display the physiological parameters or other information about the system. In the embodiment shown, monitor 14 may also include a speaker 22 to provide an audible sound that may be used in various other embodiments, such as for example, sounding an audible alarm in the event that a patient's physiological parameters are not within a predefined normal range.

In an embodiment, sensor 12, or the sensor array, may be communicatively coupled to monitor 14 via a cable 24. However, in other embodiments, a wireless transmission device (not shown) or the like may be used instead of or in addition to cable 24.

In the illustrated embodiment, pulse oximetry system 10 may also include a multi-parameter patient monitor 26. The monitor may be cathode ray tube type, a flat panel display (as shown) such as a liquid crystal display (LCD) or a plasma display, or any other type of monitor now known or later developed. Multi-parameter patient monitor 26 may be configured to calculate physiological parameters and to provide a display 28 for information from monitor 14 and from other medical monitoring devices or systems (not shown). For example, multiparameter patient monitor 26 may be configured to display an estimate of a patient's blood oxygen saturation generated by pulse oximetry monitor 14 (referred to as an “SpO₂” measurement), pulse rate information from monitor 14 and blood pressure from a blood pressure monitor (not shown) on display 28.

Monitor 14 may be communicatively coupled to multi-parameter patient monitor 26 via a cable 32 or 34 that is coupled to a sensor input port or a digital communications port, respectively and/or may communicate wirelessly (not shown). In addition, monitor 14 and/or multi-parameter patient monitor 26 may be coupled to a network to enable the sharing of information with servers or other workstations (not shown). Monitor 14 may be powered by a battery (not shown) or by a conventional power source such as a wall outlet.

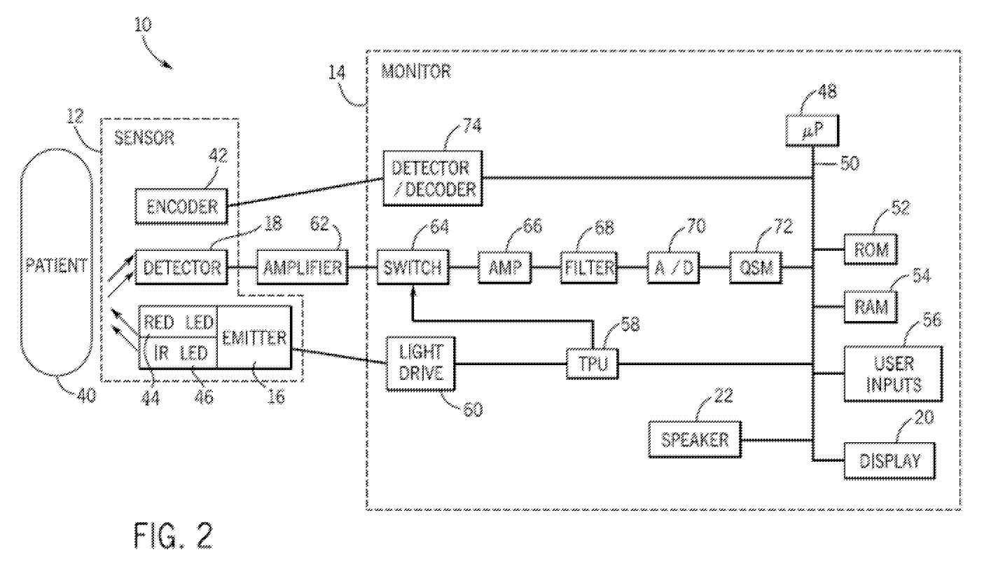

FIG. 2 is a block diagram of a pulse oximetry system, such as pulse oximetry system 10 of FIG. 1, which may be coupled to a patient 40 in accordance with an embodiment. Certain illustrative components of sensor 12 and monitor 14 are illustrated in FIG. 2. Sensor 12 may include emitter 16, detector 18, and encoder 42. In the embodiment shown, emitter 16 may be configured to emit at least two wavelengths of light (e.g., RED and IR) into a patient's tissue 40. Hence, emitter 16 may include a RED light emitting light source such as RED light emitting diode (LED) 44 and an IR light emitting light source such as IR LED 46 for emitting light into the patient's tissue 40 at the wavelengths used to calculate the patient's physiological parameters. In one embodiment, the RED wavelength may be between about 600 nm and about 700 nm, and the IR wavelength may be between about 800 nm and about 1000 nm. In embodiments where a sensor array is used in place of single sensor, each sensor may be configured to emit a single wavelength. For example, a first sensor emits only a RED light while a second only emits an IR light.

It will be understood that, as used herein, the term “light” may refer to energy produced by radiative sources and may include one or more of ultrasound, radio, microwave, millimeter wave, infrared, visible, ultraviolet, gamma ray or X-ray electromagnetic radiation. As used herein, light may also include any wavelength within the radio, microwave, infrared, visible, ultraviolet, or X-ray spectra, and that any suitable wavelength of electromagnetic radiation may be appropriate for use with the present techniques. Detector 18 may be chosen to be specifically sensitive to the chosen targeted energy spectrum of the emitter 16.

In an embodiment, detector 18 may be configured to detect the intensity of light at the RED and IR wavelengths. Alternatively, each sensor in the array may be configured to detect an intensity of a single wavelength. In operation, light may enter detector 18 after passing through the patient's tissue 40. Detector 18 may convert the intensity of the received light into an electrical signal. The light intensity is directly related to the absorbance and/or reflectance of light in the tissue 40. That is, when more light at a certain wavelength is absorbed or reflected, less light of that wavelength is received from the tissue by the detector 18. After converting the received light to an electrical signal, detector 18 may send the signal to monitor 14, where physiological parameters may be calculated based on the absorption of the RED and IR wavelengths in the patient's tissue 40. An example of a device configured to perform such calculations is the Model N600x pulse oximeter available from Nellcor Puritan Bennett LLC.

In an embodiment, encoder 42 may contain information about sensor 12, such as what type of sensor it is (e.g., whether the sensor is intended for placement on a forehead or digit) and the wavelengths of light emitted by emitter 16. This information may be used by monitor 14 to select appropriate algorithms, lookup tables and/or calibration coefficients stored in monitor 14 for calculating the patient's physiological parameters.

Encoder 42 may contain information specific to patient 40, such as, for example, the patient's age, weight, and diagnosis. This information may allow monitor 14 to determine, for example, patient-specific threshold ranges in which the patient's physiological parameter measurements should fall and to enable or disable additional physiological parameter algorithms. Encoder 42 may, for instance, be a coded resistor which stores values corresponding to the type of sensor 12 or the type of each sensor in the sensor array, the wavelengths of light emitted by emitter 16 on each sensor of the sensor array, and/or the patient's characteristics. In another embodiment, encoder 42 may include a memory on which one or more of the following information may be stored for communication to monitor 14: the type of the sensor 12; the wavelengths of light emitted by emitter 16; the particular wavelength each sensor in the sensor array is monitoring; a signal threshold for each sensor in the sensor array; any other suitable information; or any combination thereof.

In an embodiment, signals from detector 18 and encoder 42 may be transmitted to monitor 14. In the embodiment shown, monitor 14 may include a general-purpose microprocessor 48 connected to an internal bus 50. Microprocessor 48 may be adapted to execute software, which may include an operating system and one or more applications, as part of performing the functions described herein. Also connected to bus 50 may be a read-only memory (ROM) 52, a random access memory (RAM) 54, user inputs 56, display 20, and speaker 22.

RAM 54 and ROM 52 are illustrated by way of example, and not limitation. Any suitable computer-readable media may be used in the system for data storage. Computer-readable media are capable of storing information that can be interpreted by microprocessor 48. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system.

In the embodiment shown, a time processing unit (TPU) 58 may provide timing control signals to a light drive circuitry 60, which may control when emitter 16 is illuminated and multiplexed timing for the RED LED 44 and the IR LED 46. TPU 58 may also control the gating-in of signals from detector 18 through an amplifier 62 and a switching circuit 64. These signals are sampled at the proper time, depending upon which light source is illuminated. The received signal from detector 18 may be passed through an amplifier 66, a low pass filter 68, and an analog-to-digital converter 70. The digital data may then be stored in a queued serial module (QSM) 72 (or buffer) for later downloading to RAM 54 as QSM 72 fills up. In one embodiment, there may be multiple separate parallel paths having amplifier 66, filter 68, and A/D converter 70 for multiple light wavelengths or spectra received.

In an embodiment, microprocessor 48 may determine the patient's physiological parameters, such as SpO₂ and pulse rate, using various algorithms and/or look-up tables based on the value of the received signals and/or data corresponding to the light received by detector 18, Signals corresponding to information about patient 40, and particularly about the intensity of light emanating from a patient's tissue over time, may be transmitted from encoder 42 to a decoder 74. These signals may include, for example, encoded information relating to patient characteristics. Decoder 74 may translate these signals to enable the microprocessor to determine the thresholds based on algorithms or look-up tables stored in ROM 52. User inputs 56 may be used to enter information about the patient, such as age, weight, height, diagnosis, medications, treatments, and so forth. In an embodiment, display 20 may exhibit a list of values which may generally apply to the patient, such as, for example, age ranges or medication families, which the user may select using user inputs 56.

The optical signal through the tissue can be degraded by noise, among other sources. One source of noise is ambient light that reaches the light detector. Another source of noise is electromagnetic coupling from other electronic instruments. Movement of the patient also introduces noise and affects the signal. For example, the contact between the detector and the skin, or the emitter and the skin, can be temporarily disrupted when movement causes either to move away from the skin. In addition, because blood is a fluid, it responds differently than the surrounding tissue to inertial effects, thus resulting in momentary changes in volume at the point to which the oximeter probe is attached.

Noise (e.g., from patient movement) can degrade a pulse oximetry signal relied upon by a physician, without the physician's awareness. This is especially true if the monitoring of the patient is remote, the motion is too small to be observed, or the doctor is watching the instrument or other parts of the patient, and not the sensor site. Processing pulse oximetry (i.e., PPG) signals may involve operations that reduce the amount of noise present in the signals or otherwise identify noise components in order to prevent them from affecting measurements of physiological parameters derived from the PPG signals.

It will be understood that the present disclosure is applicable to any suitable signals and that PPG signals are used merely for illustrative purposes. Those skilled in the art will recognize that the present disclosure has wide applicability to other signals including, but not limited to other biosignals (e.g., electrocardiogram, electroencephalogram, electrogastrogram, electromyogram, heart rate signals, pathological sounds, ultrasound, or any other suitable biosignal), dynamic signals, non-destructive testing signals, condition monitoring signals, fluid signals, geophysical signals, astronomical signals, electrical signals, financial signals including financial indices, sound and speech signals, chemical signals, meteorological signals including climate signals, and/or any other suitable signal, and/or any combination thereof.

In one embodiment, a PPG signal may be transformed using a continuous wavelet transform. Information derived from the transform of the PPG signal (i.e., in wavelet space) may be used to provide measurements of one or more physiological parameters.

The continuous wavelet transform of a signal x(t) in accordance with the present disclosure may be defined as

$$T ( a , b ) = \frac{1}{\sqrt{a}} {\int }_{- ∞}^{+ ∞} x ( t ) {ψ}^{*} ( \frac{t - b}{a} ) d t \qquad ( 9 )$$

where ψ*(t) is the complex conjugate of the wavelet function ψ(t), a is the dilation parameter of the wavelet and b is the location parameter of the wavelet. The transform given by equation (9) may be used to construct a representation of a signal on a transform surface. The transform may be regarded as a time-scale representation. Wavelets are composed of a range of frequencies, one of which may be denoted as the characteristic frequency of the wavelet, where the characteristic frequency associated with the wavelet is inversely proportional to the scale a. One example of a characteristic frequency is the dominant frequency. Each scale of a particular wavelet may have a different characteristic frequency. The underlying mathematical detail required for the implementation within a time-scale can be found, for example, in Paul S. Addison, The Illustrated Wavelet Transform Handbook (Taylor & Francis Group 2002), which is hereby incorporated by reference herein in its entirety.

The continuous wavelet transform decomposes a signal using wavelets, which are generally highly localized in time. The continuous wavelet transform may provide a higher resolution relative to discrete transforms, thus providing the ability to garner more information from signals than typical frequency transforms such as Fourier transforms (or any other spectral techniques) or discrete wavelet transforms. Continuous wavelet transforms allow for the use of a range of wavelets with scales spanning the scales of interest of a signal such that small scale signal components correlate well with the smaller scale wavelets and thus manifest at high energies at smaller scales in the transform. Likewise, large scale signal components correlate well with the larger scale wavelets and thus manifest at high energies at larger scales in the transform. Thus, components at different scales may be separated and extracted in the wavelet transform domain. Moreover, the use of a continuous range of wavelets in scale and time position allows for a higher resolution transform than is possible relative to discrete techniques.

In addition, transforms and operations that convert a signal or any other type of data into a spectral (i.e., frequency) domain necessarily create a series of frequency transform values in a two-dimensional coordinate system where the two dimensions may be frequency and, for example, amplitude. For example, any type of Fourier transform would generate such a two-dimensional spectrum. In contrast, wavelet transforms, such as continuous wavelet transforms, are required to be defined in a three-dimensional coordinate system and generate a surface with dimensions of time, scale and, for example, amplitude. Hence, operations performed in a spectral domain cannot be performed in the wavelet domain; instead the wavelet surface must be transformed into a spectrum (i.e., by performing an inverse wavelet transform to convert the wavelet surface into the time domain and then performing a spectral transform from the time domain). Conversely, operations performed in the wavelet domain cannot be performed in the spectral domain; instead a spectrum must first be transformed into a wavelet surface (i.e. by performing an inverse spectral transform to convert the spectral domain into the time domain and then performing a wavelet transform from the time domain). Nor does a cross-section of the three-dimensional wavelet surface along, for example, a particular point in time equate to a frequency spectrum upon which spectral-based techniques may be used. At least because wavelet space includes a time dimension, spectral techniques and wavelet techniques are not interchangeable. It will be understood that converting a system that relies on spectral domain processing to one that relies on wavelet space processing would require significant and fundamental modifications to the system in order to accommodate the wavelet space processing (e.g., to derive a representative energy value for a signal or part of a signal requires integrating twice, across time and scale, in the wavelet domain while, conversely, one integration across frequency is required to derive a representative energy value from a spectral domain). As a further example, to reconstruct a temporal signal requires integrating twice, across time and scale, in the wavelet domain while, conversely, one integration across frequency is required to derive a temporal signal from a spectral domain. It is well known in the art that, in addition to or as an alternative to amplitude, parameters such as energy density, modulus, phase, among others may all be generated using such transforms and that these parameters have distinctly different contexts and meanings when defined in a two-dimensional frequency coordinate system rather than a three-dimensional wavelet coordinate system. For example, the phase of a Fourier system is calculated with respect to a single origin for all frequencies while the phase for a wavelet system is unfolded into two dimensions with respect to a wavelet's location (often in time) and scale.

The energy density function of the wavelet transform, the scalogram, is defined as

S(a,b)=|T(a,b)|² (10)

where ‘∥’ is the modulus operator. The scalogram may be resealed for useful purposes. One common rescaling is defined as

$${S}_{R} ( a , b ) = \frac{{\vert T ( a , b ) \vert}^{2}}{a} \qquad ( 11 )$$

and is useful for defining ridges in wavelet space when, for example, the Morlet wavelet is used. Ridges are defined as the locus of points of local maxima in the plane. Any reasonable definition of a ridge may be employed in the method. Also included as a definition of a ridge herein are paths displaced from the locus of the local maxima. A ridge associated with only the locus of points of local maxima in the plane are labeled a “maxima ridge”.

For implementations requiring fast numerical computation, the wavelet transform may be expressed as an approximation using Fourier transforms. Pursuant to the convolution theorem, because the wavelet transform is the cross-correlation of the signal with the wavelet function, the wavelet transform may be approximated in terms of an inverse FFT of the product of the Fourier transform of the signal and the Fourier transform of the wavelet for each required a scale and then multiplying the result by \sqrt{a}.

In the discussion of the technology which follows herein, the “scalogram” may be taken to include all suitable forms of resealing including, but not limited to, the original unsealed wavelet representation, linear resealing, any power of the modulus of the wavelet transform, or any other suitable resealing. In addition, for purposes of clarity and conciseness, the term “scalogram” shall be taken to mean the wavelet transform, T(a,b) itself, or any part thereof. For example, the real part of the wavelet transform, the imaginary part of the wavelet transform, the phase of the wavelet transform, any other suitable part of the wavelet transform, or any combination thereof is intended to be conveyed by the term “scalogram”.

A scale, which may be interpreted as a representative temporal period, may be converted to a characteristic frequency of the wavelet function. The characteristic frequency associated with a wavelet of arbitrary a scale is given by

$$f = \frac{{f}_{c}}{a} \qquad ( 12 )$$

where f_{c}, the characteristic frequency of the mother wavelet (i.e., at a=1), becomes a scaling constant and f is the representative or characteristic frequency for the wavelet at arbitrary scale a.

Any suitable wavelet function may be used in connection with the present disclosure. One of the most commonly used complex wavelets, the Morlet wavelet, is defined as:

ψ(t)=π^{-1/4}(e^{i2πf}^{₀}^{t}−e^{-(2πf}^{₀}⁾^{²}²)^{-t}^{²}^{/2} (13)

where f₀ is the central frequency of the mother wavelet. The second term in the parenthesis is known as the correction term, as it corrects for the non-zero mean of the complex sinusoid within the Gaussian window. In practice, it becomes negligible for values of f₀>>0 and can be ignored, in which case, the Morlet wavelet can be written in a simpler form as

$$ψ ( t ) = \frac{1}{{π}^{1 / 4}} {e}^{i 2 π {f}_{0} t} {e}^{- {t}^{2} / 2} \qquad ( 14 )$$

This wavelet is a complex wave within a scaled Gaussian envelope. While both definitions of the Morlet wavelet are included herein, the function of equation (14) is not strictly a wavelet as it has a non-zero mean (i.e., the zero frequency term of its corresponding energy spectrum is non-zero). However, it will be recognized by those skilled in the art that equation (14) may be used in practice with f₀>>0 with minimal error and is included (as well as other similar near wavelet functions) in the definition of a wavelet herein. A more detailed overview of the underlying wavelet theory, including the definition of a wavelet function, can be found in the general literature. Discussed herein is how wavelet transform features may be extracted from the wavelet decomposition of signals. For example, wavelet decomposition of PPG signals may be used to provide clinically useful information within a medical device.

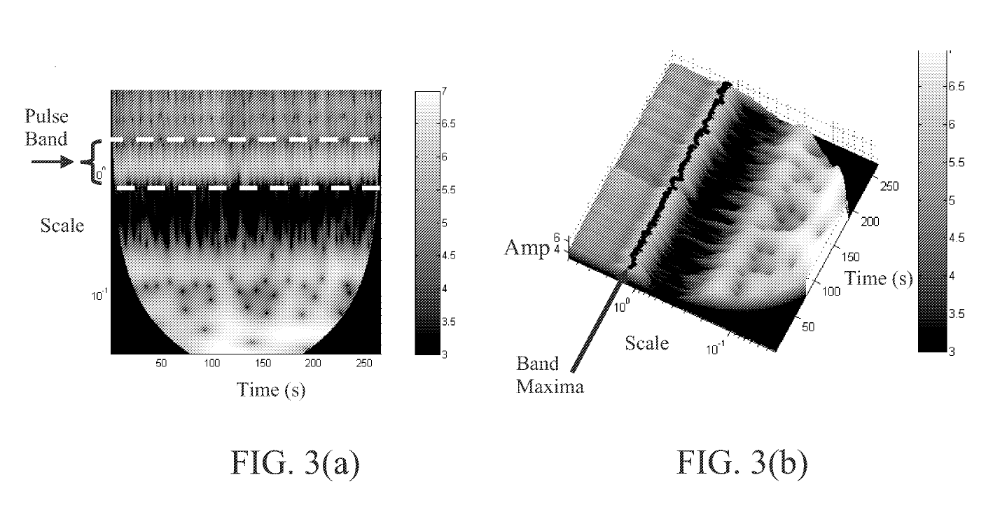

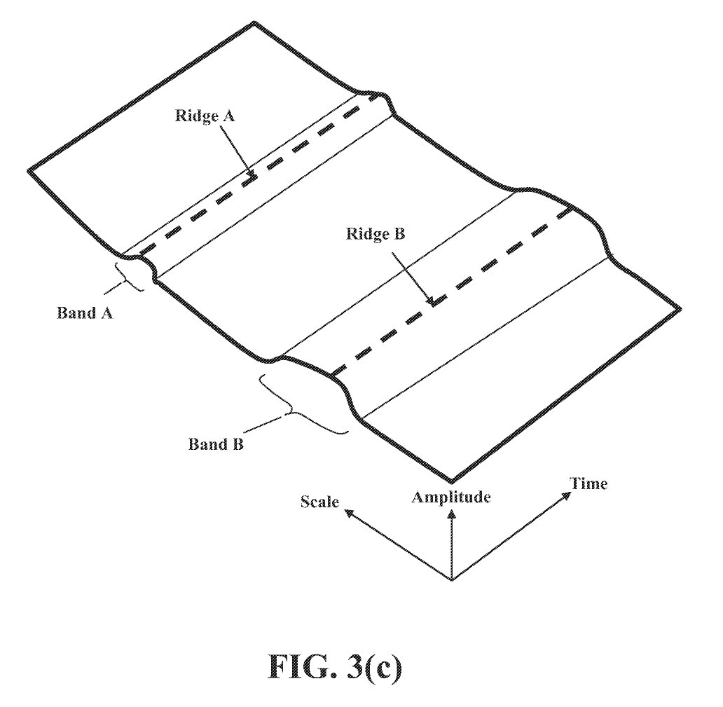

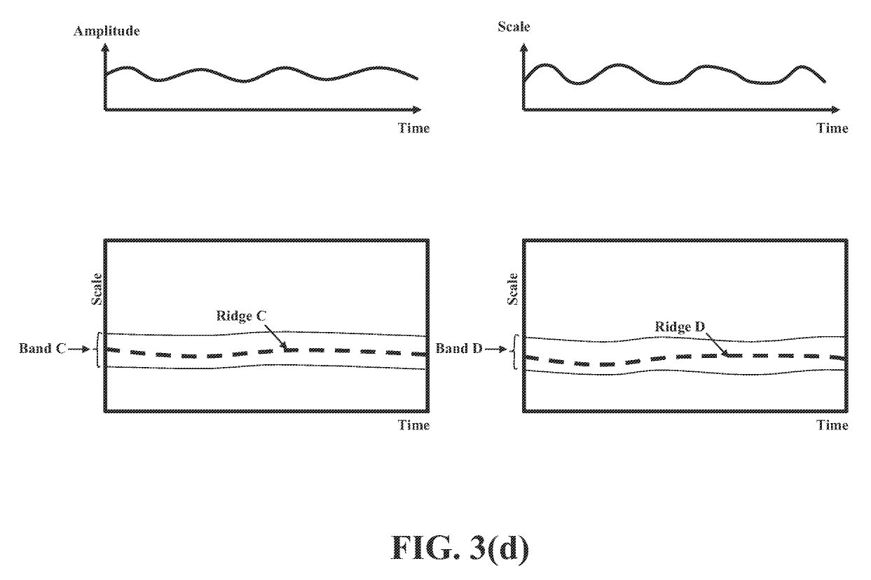

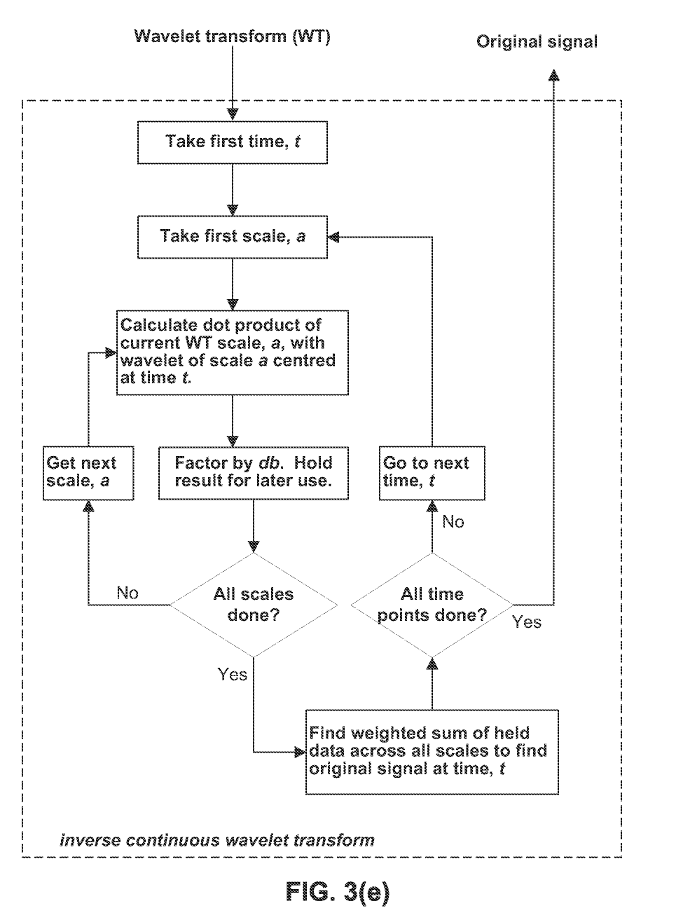

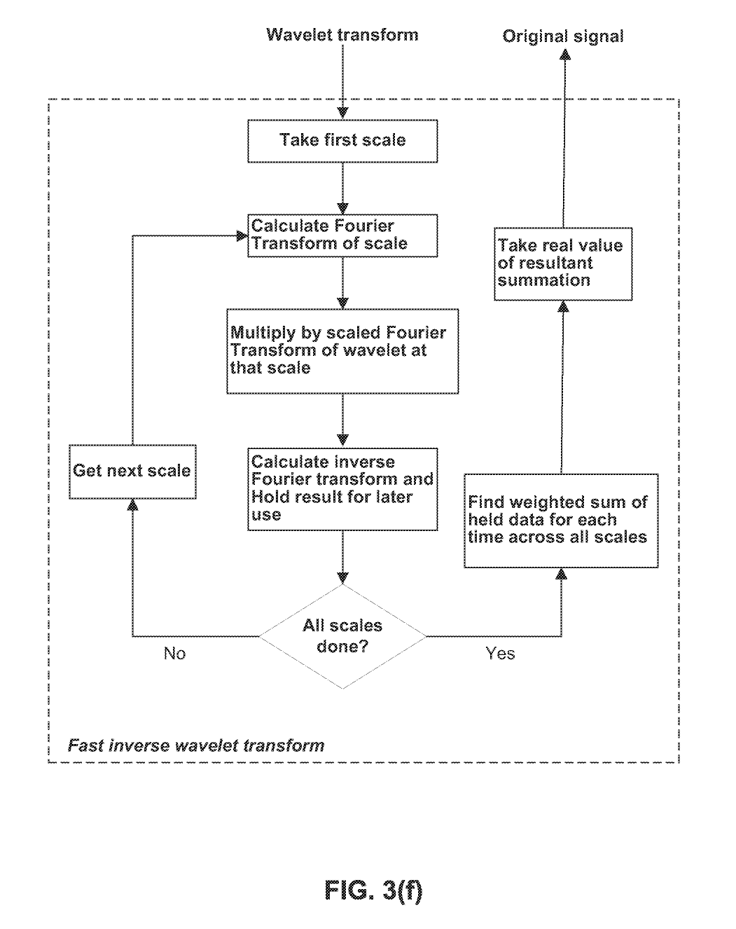

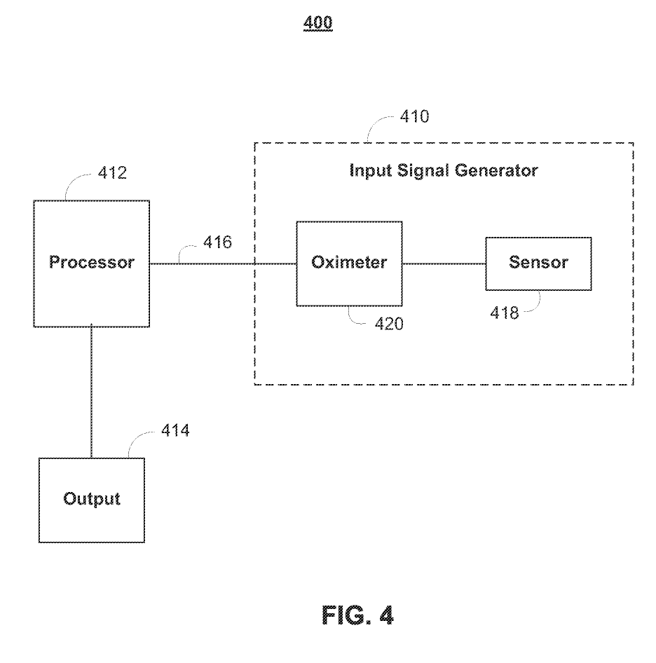

Pertinent repeating features in a signal give rise to a time-scale band in wavelet space or a resealed wavelet space. For example, the pulse component of a PPG signal produces a dominant band in wavelet space at or around the pulse frequency. FIGS. 3(a) and (b) show two views of an illustrative scalogram derived from a PPG signal, according to an embodiment. The figures show an example of the band caused by the pulse component in such a signal. The pulse band is located between the dashed lines in the plot of FIG. 3(a). The band is formed from a series of dominant coalescing features across the scalogram. This can be clearly seen as a raised band across the transform surface in FIG. 3(b) located within the region of scales indicated by the arrow in the plot (corresponding to 60 beats per minute). The maxima of this band with respect to scale is the ridge. The locus of the ridge is shown as a black curve on top of the band in FIG. 3(b). By employing a suitable resealing of the scalogram, such as that given in equation (11), the ridges found in wavelet space may be related to the instantaneous frequency of the signal. In this way, the pulse rate may be obtained from the PPG signal. Instead of resealing the scalogram, a suitable predefined relationship between the scale obtained from the ridge on the wavelet surface and the actual pulse rate may also be used to determine the pulse rate.

By mapping the time-scale coordinates of the pulse ridge onto the wavelet phase information gained through the wavelet transform, individual pulses may be captured. In this way, both times between individual pulses and the timing of components within each pulse may be monitored and used to detect heart beat anomalies, measure arterial system compliance, or perform any other suitable calculations or diagnostics. Alternative definitions of a ridge may be employed. Alternative relationships between the ridge and the pulse frequency of occurrence may be employed.

As discussed above, pertinent repeating features in the signal give rise to a time-scale band in wavelet space or a resealed wavelet space. For a periodic signal, this band remains at a constant scale in the time-scale plane. For many real signals, especially biological signals, the band may be non-stationary; varying in scale, amplitude, or both over time. FIG. 3(c) shows an illustrative schematic of a wavelet transform of a signal containing two pertinent components leading to two bands in the transform space, according to an embodiment. These bands are labeled band A and band B on the three-dimensional schematic of the wavelet surface. In this embodiment, the band ridge is defined as the locus of the peak values of these bands with respect to scale. For purposes of discussion, it may be assumed that band B contains the signal information of interest. This will be referred to as the “primary band”. In addition, it may be assumed that the system from which the signal originates, and from which the transform is subsequently derived, exhibits some form of coupling between the signal components in band A and band B. When noise or other erroneous features are present in the signal with similar spectral characteristics of the features of band B then the information within band B can become ambiguous (i.e., obscured, fragmented or missing). In this case, the ridge of band A may be followed in wavelet space and extracted either as an amplitude signal or a scale signal which will be referred to as the “ridge amplitude perturbation” (RAP) signal and the “ridge scale perturbation” (RSP) signal, respectively. The RAP and RSP signals may be extracted by projecting the ridge onto the time-amplitude or time-scale planes, respectively. The top plots of FIG. 3(d) show a schematic of the RAP and RSP signals associated with ridge A in FIG. 3(c). Below these RAP and RSP signals are schematics of a further wavelet decomposition of these newly derived signals. This secondary wavelet decomposition allows for information in the region of band B in FIG. 3(c) to be made available as band C and band D. The ridges of bands C and D may serve as instantaneous time-scale characteristic measures of the signal components causing bands C and D. This technique, which will be referred to herein as secondary wavelet feature decoupling (SWFD), may allow information concerning the nature of the signal components associated with the underlying physical process causing the primary band B (FIG. 3(c)) to be extracted when band B itself is obscured in the presence of noise or other erroneous signal features.

In some instances, an inverse continuous wavelet transform may be desired, such as when modifications to a scalogram (or modifications to the coefficients of a transformed signal) have been made in order to, for example, remove artifacts. In one embodiment, there is an inverse continuous wavelet transform which allows the original signal to be recovered from its wavelet transform by integrating over all scales and locations, a and b:

$$x ( t ) = \frac{1}{{C}_{g}} {\int }_{- ∞}^{∞} {\int }_{0}^{∞} T ( a , b ) \frac{1}{\sqrt{a}} ψ ( \frac{t - b}{a} ) \frac{d a d b}{{a}^{2}} \qquad ( 15 )$$

which may also be written as:

$$x ( t ) = \frac{1}{{C}_{g}} {\int }_{- ∞}^{∞} {\int }_{0}^{∞} T ( a , b ) {ψ}_{a , b} ( t ) \frac{d a d b}{{a}^{2}} \qquad ( 16 )$$

where C_{g} is a scalar value known as the admissibility constant. It is wavelet type dependent and may be calculated from:

$${C}_{g} = {\int }_{0}^{∞} \frac{{\vert \hat{ψ} ( f ) \vert}^{2}}{f} d f \qquad ( 17 )$$

FIG. 3(e) is a flow chart of illustrative steps that may be taken to perform an inverse continuous wavelet transform in accordance with the above discussion. An approximation to the inverse transform may be made by considering equation (15) to be a series of convolutions across scales. It shall be understood that there is no complex conjugate here, unlike for the cross correlations of the forward transform. As well as integrating over all of a and b for each time t, this equation may also take advantage of the convolution theorem which allows the inverse wavelet transform to be executed using a series of multiplications. FIG. 3(f) is a flow chart of illustrative steps that may be taken to perform an approximation of an inverse continuous wavelet transform. It will be understood that any other suitable technique for performing an inverse continuous wavelet transform may be used in accordance with the present disclosure.

FIG. 4 is an illustrative continuous wavelet processing system in accordance with an embodiment. In this embodiment, input signal generator 410 generates an input signal 416. As illustrated, input signal generator 410 may include oximeter 420 coupled to sensor 418, which may provide as input signal 416, a PPG signal. It will be understood that input signal generator 410 may include any suitable signal source, signal generating data, signal generating equipment, or any combination thereof to produce signal 416. Signal 416 may be any suitable signal or signals, such as, for example, biosignals (e.g., electrocardiogram, electroencephalogram, electrogastrogram, electromyogram, heart rate signals, pathological sounds, ultrasound, or any other suitable biosignal), dynamic signals, non-destructive testing signals, condition monitoring signals, fluid signals, geophysical signals, astronomical signals, electrical signals, financial signals including financial indices, sound and speech signals, chemical signals, meteorological signals including climate signals, and/or any other suitable signal, and/or any combination thereof.

In this embodiment, signal 416 may be coupled to processor 412. Processor 412 may be any suitable software, firmware, and/or hardware, and/or combinations thereof for processing signal 416. For example, processor 412 may include one or more hardware processors (e.g., integrated circuits), one or more software modules, computer-readable media such as memory, firmware, or any combination thereof. Processor 412 may, for example, be a computer or may be one or more chips (i.e., integrated circuits). Processor 412 may perform the calculations associated with the continuous wavelet transforms of the present disclosure as well as the calculations associated with any suitable interrogations of the transforms. Processor 412 may perform any suitable signal processing of signal 416 to filter signal 416, such as any suitable band-pass filtering, adaptive filtering, closed-loop filtering, and/or any other suitable filtering, and/or any combination thereof.

Processor 412 may be coupled to one or more memory devices (not shown) or incorporate one or more memory devices such as any suitable volatile memory device (e.g., RAM, registers, etc.), non-volatile memory device (e.g., ROM, EPROM, magnetic storage device, optical storage device, flash memory, etc.), or both. The memory may be used by processor 412 to, for example, store data corresponding to a continuous wavelet transform of input signal 416, such as data representing a scalogram. In one embodiment, data representing a scalogram may be stored in RAM or memory internal to processor 412 as any suitable three-dimensional data structure such as a three-dimensional array that represents the scalogram as energy levels in a time-scale plane. Any other suitable data structure may be used to store data representing a scalogram.

Processor 412 may be coupled to output 414. Output 414 may be any suitable output device such as, for example, one or more medical devices (e.g., a medical monitor that displays various physiological parameters, a medical alarm, or any other suitable medical device that either displays physiological parameters or uses the output of processor 412 as an input), one or more display devices (e.g., monitor, PDA, mobile phone, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

It will be understood that system 400 may be incorporated into system 10 (FIGS. 1 and 2) in which, for example, input signal generator 410 may be implemented as parts of sensor 12 and monitor 14 and processor 412 may be implemented as part of monitor 14. Processor 412 (FIG. 4) or microprocessor 48 (FIG. 2) may be used to process physiological signals, such as PPG signals, to calculate physiological parameters associated with the signals. The signals may be provided in a patient monitoring scenario using, one or more sensors 12 (FIG. 1), an input signal generator 410 (FIG. 4), or other device. The signals may include PPG signals or other types of signals that are appropriate for calculating physiological parameters using various techniques.

Systems 400 (FIG. 4) and 10 (FIGS. 1 and 2) may use one or more physiological signals to calculate physiological parameters such as pulse rate, blood pressure, respiration rate, respiration effort, oxygen saturation, or other suitable physiological parameters. The physiological parameters may be determined using various techniques, and combinations of techniques including CWT techniques (e.g., using one or more scalograms derived from one or more PPG signals) and non-CWT techniques.

Pulse Rate

In an embodiment, systems 400 (FIG. 4) and 10 (FIGS. 1 and 2) may use CWT and non-CWT techniques for calculating pulse rate. As discussed above, the pulse component of a PPG signal may produce a dominant band in a scalogram. The pulse rate may be calculated, for example, using a CWT technique by generating a scalogram from a PPG signal, following or identifying the ridge of the pulse band, identifying a scale corresponding to the ridge, and selecting the pulse rate to be the characteristic frequency of the identified scale.

The pulse rate may also be calculated using non-CWT techniques such as time based or spectral techniques. For example, the time based PPG signal may be evaluated to detect peaks corresponding to heart beats. The detected heart beats may then be used to determine the pulse rate. Time based techniques for detecting heart beats and calculating the pulse rate are described in detail in U.S. patent application Ser. No. ______, filed Sep. 30, 2008, entitled “Systems And Methods For Detecting Pulses,” (Att. Docket No.: H-RM-01193-1 (COV-9-01)), the content of which is hereby incorporated by reference in its entirety.

The Fourier transform is another non-CWT technique that may also be used to calculate the pulse rate. For example, a segment of the PPG signal may be transformed using the Fourier transform. The frequency corresponding to a spectral peak (e.g., the maximum peak) in the transformed signal may be selected as the pulse rate. Any other suitable non-CWT technique may also be used to calculate pulse rate.

Blood Pressure

Systems 400 (FIG. 4) and 10 (FIGS. 1 and 2) may also use CWT and non-CWT techniques for calculating blood pressure. One CWT technique for calculating blood pressure technique may include generating a scalogram from a PPG signal and using a wavelet phase to detect a differential pulse transit time (DPTT) timings using wavelet phase. A real value wavelet may also be used to find a scale dependent DPTT value. Detection of DPTT timing may be used in conjunction with modulus maxima techniques, or other techniques. Another CWT technique for calculating blood pressure may include generating a scalogram from a PPG signal and measuring an area for a wavelet space. The boundaries of the area may be identified by identifying one or more scales and determining a make up of an area parameter resolved across scales. Other CWT techniques for calculating blood pressure may also be used.

Non-CWT techniques for calculating blood pressure may include, for example, using an inflatable blood pressure cuff Other Non-CWT techniques may also be used.

Respiration Rate

Respiration rate may also be calculated using CWT and non-CWT techniques. The respiration component of a PPG signal may produce a band in a scalogram similar to the pulse band. Therefore, the respiration rate may be calculated, for example, using a CWT technique by generating a scalogram from a PPG signal, following or identifying the ridge of the respiration band (e.g., located at scales lower than the scales where the typically more dominant pulse band occurs), identifying a scale corresponding to the ridge, and selecting the respiration rate to be the characteristic frequency of the identified scale. The respiration component of the PPG signal may also cause modulations of the pulse band. Thus, the respiration rate may also be calculated by performing a secondary wavelet decomposition of modulations (e.g., of RAP and RSP signals) of the pulse band. These and other CWT techniques for calculating respiration rate are described in detail in Addison et al. U.S. Pat. No. 7,035,679, Addison et al. U.S. Patent Publication No. 2006/0258921, and U.S. patent application Ser. No. ______, filed Oct. 3, 2008, entitled “Systems And Methods For Ridge Selection In Scalograms Of Signals,” (Att. Docket No.: H-RM-01197-1 (COV-2-01)), each of which is hereby incorporated by reference herein in its entirety.

The respiration rate may also be calculated using non-CWT techniques. For example, a segment of the PPG signal may be transformed using the Fourier transform. The frequency corresponding to a spectral peak (e.g., a local maximum peak) in the transformed signal may be selected as the respiration rate. Any other suitable non-CWT technique may also be used to calculate respiration rate.

Respiration Effort

Respiration Effort may also be calculated using CWT and non-CWT techniques. One CWT technique for calculating respiration effort may include detecting and analyzing features of a breathing band or respiration band in a scalogram. For example, a scalogram may be generated based on a PPG signal. One or more features of the scalogram may be used to determine respiration effort. Such features may include a measure of strength of a repetitive feature in a signal, changes in features of a signal used to generate the scalogram. For example, a breathing band or respiration band and/or its features may occur at a frequency scale of a breathing frequency. Features within the breathing band or other bands on the scalogram (e.g., energy, amplitude, or modulation) may result from changes in breathing effort and therefore may be correlated with the patient's breathing effort. This technique is further described in U.S. patent application Ser. No. ______, filed Sep. 30, 2008, entitled, entitled “SYSTEMS AND METHODS FOR DETERMINING EFFORT,” (Att. Docket No. H-RM-01194-2 (COV-4-02)), and its priority applications: U.S. Provisional Application No. 61/077,097, filed Jun. 30, 2008 and U.S. Provisional Application No. 61/077,130, filed Jun. 30, 2008, each of which is hereby incorporated by reference herein in their entireties.

Non-CWT techniques for calculating respiration effort may include analyzing a signal amplitude for any non-CWT technique. In addition, a Fourier transform may be performed on a PPG signal or segment of the signal. A change in a Fourier frequency energy for a respiration rate may be used to identify a change in breathing effort. Any other suitable non-CWT technique may also be used to calculate respiration effort.

Blood Oxygen Saturation

Systems 400 (FIG. 4) and 10 (FIGS. 1 and 2) may also use CWT and non-CWT techniques for calculating oxygen saturation. Oxygen saturation may be determined, for example, by computing the ratio of points on two scalograms (e.g., at the location of the pulse band) and using, for example, a lookup table or an equation to obtain oxygen saturation. Another continuous wavelet transform-based technique for calculating blood oxygen saturation involves generating a Lissajous figure in which the transformed red and infrared signals (i.e., using a continuous wavelet transform) are plotted with respect to one another. These CWT techniques and other CWT techniques for determining oxygen saturation are described in detail in Addison et al. U.S. Patent App. Pub. No. 2006/0258921. Another exemplary CWT technique that may be used in accordance with this disclosure is described in U.S. patent application Ser. No. ______, filed Oct. 3, 2008, entitled, entitled “METHODS AND SYSTEMS FOR FILTERING A SIGNAL ACCORDING TO A SIGNAL MODEL AND CONTINUOUS WAVELET TRANSFORM TECHNIQUES,” (Att. Docket No. H-RM01256-1 (COV-20-01)), which is hereby incorporated by reference herein in its entirety. This CWT technique includes generating a plurality of possible values in accordance with a signal model and determining which of the values has a highest energy level (e.g., to minimize correlation), and other techniques.

Oxygen saturation may also be calculated using time based and spectral techniques (i.e., non-CWT techniques). For example, blood oxygen saturation may be calculated using the “ratio of ratios” time based technique discussed above. This technique generally analyzes the change in the red PPG signal over the changes in the infrared signal and uses the computed ratio in, for example, a lookup table or equation to calculate oxygen saturation. Blood oxygen saturation may also be calculated by using a Fourier transform technique. For example, Fourier transforms may be performed on the red and infrared PPG signals. The ratio of peaks in the transformed signals may similarly be used in, for example, a lookup table or equation to calculate oxygen saturation.

The foregoing CWT and non-CWT techniques for calculating physiological parameters may be used in combination for determining a final value for the physiological parameter. For example, a physiological parameter (e.g., oxygen saturation) may be calculated using a CWT technique and a non-CWT technique. The results may be combined, for example, by selecting one of the two results (e.g., the one that is closest to an expected or historical value) or by taking an average or weighted average to obtain an improved or desired value for the calculated physiological parameter.

The foregoing CWT and non-CWT techniques may also be used together for calculating physiological parameters. For example, non-CWT techniques may be less computationally intensive than CWT techniques. The use of non-CWT techniques (e.g., Fourier transform techniques) may thus quickly or easily identify frequencies or frequency ranges that may be of interest (e.g., pulse rate or respiration rate frequencies). Based on this information, the CWT technique may then use a greater scale resolution and/or a lesser number of scales in the continuous wavelet transform to calculate physiological parameters. For example, the CWT technique may be performed at scales with characteristic frequencies corresponding to the frequencies of interest. The CWT technique may provide a more accurate calculation of the physiological parameter due to the higher resolution or may perform its calculations more quickly. As another example, a CWT technique may be used to identify a scale or range of scales that may be of interest. Based on this information, a non-CWT technique (e.g., a Fourier technique) may be performed focusing on the characteristic frequency or frequencies of the identified scale or scales.

As discussed above, CWT techniques and non-CWT techniques may be used to calculate physiological parameters based on PPG signals. In some instances, there are certain advantages to using one technique over another, or using one or more combinations of techniques. For example, certain techniques may provide more statistically or historically accurate calculations for a certain scenario. Other techniques may provide more accurate calculations for another scenario. For yet other scenarios, combinations of techniques may provide a most accurate result. The use of one or more techniques and/or combinations of techniques to provide a physiological parameter may enhance the accuracy of information relating to physiological parameters which in turn may provide improved and more reliable information in patient monitoring.

The techniques discussed herein may be provided using components as shown in FIGS. 1-2 and 4. For example, processor 412 (FIG. 4) or microprocessor 48 (FIG. 2) may be used to process physiological signals obtained using input signal generator 410 (FIG. 4), an oximeter 420 (FIG. 4), sensor 12 (FIG. 1) or other device, using CWT techniques and non-CWT techniques.

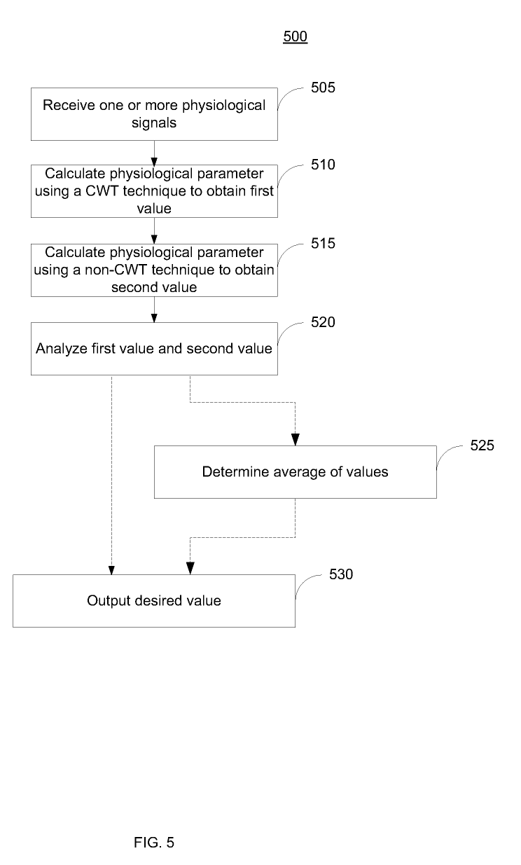

In an embodiment, a process 500 depicted in FIG. 5, may be provided to determine a desired value for use, for example, in indicating a physiological parameter in patient monitoring. Patient monitoring using an oximeter, such as sensor 12 (FIG. 1) or other device, may produce a physiological signal that may be received at step 505 by, for example, processor 412 (FIG. 4). The physiological signal may be any type of physiological signal described herein such as a PPG signal or any other suitable signal. At step 510, a calculation may be performed to obtain a value for a physiological parameter using a CWT technique. As discussed previously, the physiological parameter value may be calculated using a processor, such as processor 412 (FIG. 4), using any one or more techniques involving a continuous wavelet transform, such as by using a continuous wavelet transform to generate one or more scalograms and then analyzing one or more features in the scalograms, or other technique using a continuous wavelet transform. For example, a value for blood oxygen saturation may be determined, for example, by computing the ratio of points on two scalograms (e.g., at the location of the pulse band) and using, for example, a lookup table or an equation to obtain oxygen saturation. Other continuous wavelet transform techniques may also be used.

At step 515, a processor, such as processor 412 (FIG. 4) may calculate a physiological parameter using a technique that is not based on continuous wavelet transform to produce another value. Using the oxygen saturation example, a value may be calculated using time based or spectral techniques (e.g., a using a Fourier transform). In general, steps 510 and 515 may be designed to calculate the same type of physiological parameter using different techniques (i.e., one that is based on CWT, the other not based on CWT). The order of steps 510 and 515 is not critical, either may occur first or second. It will be understood by one of skill in the art, that steps 510 and 515 may be repeated any number of times using the same, different, or a combination of CWT and non-CWT techniques to produce multiple values.

The values obtained at steps 510 and 515 may be analyzed at step 520 to obtain a desired value for output at step 530. The analysis at step 520 may be performed using a processor, such as processor 412 (FIG. 4). The analysis may include any one or more techniques, such as comparing the values obtained at step 510 and 515 to, for example, a range of expected values for a certain physiological parameter, a range of expected values for a certain technique, patient information, historical information, noise information, statistical measures, empirical data, ranges of outlier values, or other standards. Such standards and ranges may be entered by a user, such as a health care professional using user input device 56 (FIG. 2). Standards and ranges may also be obtained by consulting a look up table of standards, which may be provided via a database accessible over a network connection, or provided locally (e.g., using memory 52, 54 (FIG. 2)).

In an example using a range of expected values, a CWT technique based value for respiration rate may be determined to not fall within an expected range of values. In this case, another value, such as a non-CWT technique based value for respiration rate that is determined to fall within the expected range of values may be determined to be a more desirable value. In another example, using patient information that indicates that the monitored patient is a neonate (rather than an adult), certain CWT or non-CWT techniques may be determined to produce more reliable results. An example of using historical information may be that multiple repeated calculations are performed based on a signal over time and when one or more values deviates from a set of historical values, such deviating values may be determined to not be desired values. In this example, values that are consistent with the set of historical values may be determined to be the desired value(s).

The analysis at step 520 may also consider quality of the signal used to calculate the value. For example, signal noise may also be considered in the analysis of the values. Since signal noise can cause a non-CWT technique derived value to be less reliable than one derived by a CWT technique, or vice-versa, the CWT technique or non-CWT technique may be determined to be a more desirable value. Such analysis may be performed, for example, using processor 412 (FIG. 4) to identify noise in the original signal or noise in the transformed signal. For example, noise in the scalogram may be identified by analyzing the amplitude in the scalogram at the scale or scales of interest (e.g., alone or in comparison to amplitudes in other regions of the scalogram). Noise in a non-CWT technique such as a Fourier transform may be identified by analyzing amplitudes at a frequency or frequencies of interest (e.g., alone or in comparison to amplitudes at different frequencies).

The analysis at step 520 may also consider one or more confidence indicators. A confidence indicator may be associated with a particular CWT or non-CWT technique and a physiological parameter. For example, a confidence indicator for using a Lissajous figure derived from two scalograms for calculating oxygen saturation may be greater than for using a spectral technique (or other non-CWT technique). Such confidence indicator may be input by a user, or stored in a look up table. A confidence indicator may also be determined in the analysis at step 520 based on comparisons of the values to the historical data, ranges of expected values, or other information. For example, for a value that does not fall within a range of expected values, its confidence indicator may be set relatively low.

At optional step 525, the values may be averaged to produce a desired value. The averaging of the values may be performed by processor 412 (FIG. 4). In some embodiments, the average is a straight average of the values. In other embodiments, a weighted average may be used. One or more weights may be assigned to each value. The one or more weights may be based on confidence indicators, historical information, noise information, patient information, empirical data, user inputted data, or other data. In an example using the confidence indicator as the basis for weighting, when a value does not fall within a range of expected values, it may assigned a low confidence indicator, which may lead to a relatively low weight being assigned to the value. In another example using historical information, for a value that is consistent with a set of historical values, a relatively high weight may be assigned to the value. In an example using patient information, certain patient details may indicate that one value or technique may be more reliable, which may correspond to a higher weighting. The average or weighted average value obtained at step 525 may be output as a desired value at step 530. The desired value may be output to, for example, output 414 (FIG. 4), or shown on a display, such as displays 28 or 20 (FIG. 1). Alternatively, if step 525 is not performed, then at step 530 one of the calculated values may be selected to be outputted as the desired value based on the analysis at step 520.

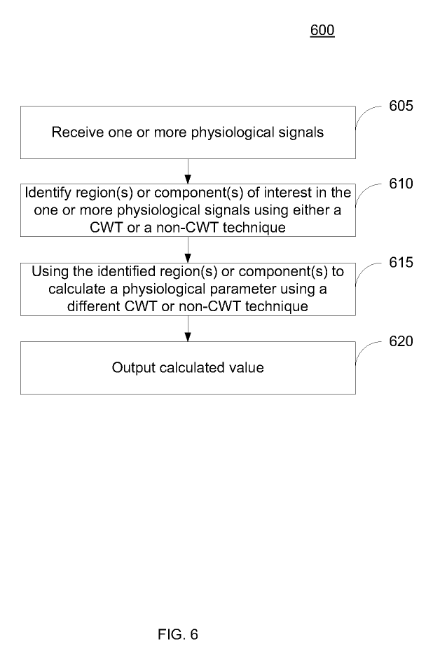

Combinations of calculation techniques may also be used to provide reliable values for patient monitoring purposes. FIG. 6 depicts a process 600 that may use a combination of continuous wavelet transform techniques and non-continuous wavelet transform techniques. Process 600 may be used to provide one or more of the values or calculations in steps 510 or 515 (FIG. 5). As shown in FIG. 6, one or more physiological signals or other signals may be received at step 605. Step 605 may generally correspond to step 505, and may include processor 412 (FIG. 4) receiving a signal from an oximeter, such as sensor 12 (FIG. 1) or other device. A CWT or non-CWT technique performed on the signal may be used to identify one or more regions or components of interest in the signal at step 610. The technique may be performed using a processor, such as processor 412 (FIG. 4) using any CWT or non-CWT technique. For example, a time based analysis (i.e., a non-CWT technique) may be performed on the received signal to identify heart beats and the corresponding pulse rate. As another example, a continuous wavelet transform may be performed on the received signal to identify a scale of interest (e.g., a scale corresponding to the pulse or respiration band).

At step 615, the identified region or components of interest may be used to calculate a physiological parameter using a different CWT or non-CWT technique. The calculation at step 615 may be performed using processor 412 (FIG. 4) or other processor. For example, oxygen saturation may be calculated using the different technique. As discussed above, a non-CWT time based technique may be used to determine pulse rate. A CWT technique may then use the pulse rate in its calculation of oxygen saturation. In one approach, the pulse rate may be used to determine what scale or range of scales are to be used in performing a continuous wavelet transform of two PPG signals. One or both scalograms generated from the two PPG signals may be analyzed to identify a ridge of the pulse band using, for example, ridge following techniques or other techniques. The ratio of amplitudes of the ridge of the pulse band in the two scalograms may be used to calculate oxygen saturation. This approach may provide improved efficiency and accuracy in generating the scalograms and in calculating the oxygen saturation. In addition to, or as an alternative approach, the pulse rate may be used to identify a scale whose characteristic frequency corresponds to the pulse rate. Oxygen saturation may be calculated by taking a ratio of amplitudes in the two scalograms at the identified scale

As another example, respiration rate may be calculated using the different technique. A CWT technique may be used on a PPG signal to generate a scalogram and identify a ridge corresponding to the breathing band. The frequency corresponding to the characteristic frequency of the scale location of the breathing band ridge may be used in a different technique (i.e., a non-CWT technique) to calculate respiration rate. A Fourier transform (i.e., a non-CWT technique) may be performed using a high resolution range of frequencies about the frequency identified using the CWT technique. The maximum amplitude in the Fourier transform may be selected as the respiration rate.

The foregoing examples are merely illustrative. Other physiological parameters (e.g., pulse rate) or any other parameters may be calculated using process 600. In addition, for each parameter that may be calculated using process 600, a non-CWT technique may be used at step 610 and a CWT technique may be used at step 620, or vice versa.

The resulting value may be output at step 620 to, for example, output 414 (FIG. 4), or shown on a display, such as displays 28 or 20 (FIG. 1).

The foregoing is merely illustrative of the principles of this disclosure and various modifications can be made by those skilled in the art without departing from the scope and spirit of the disclosure.

What is claimed is:

1. A method for determining a physiological parameter from a physiological signal, comprising:
receiving at least one physiological signal;
calculating a first value for a physiological parameter based at least in part on the at least one physiological signal using a continuous wavelet transform technique;
calculating a second value for the physiological parameter based at least in part on the at least one physiological signal using a non-continuous wavelet transform technique;
analyzing the first value and the second value; and
determining, based at least in part on the analysis of the first value and the second value, a desired value for the physiological parameter.

2. The method of claim 1 wherein the at least one physiological signal comprises a photoplethysmogram signal.

3. The method of claim 1 wherein analyzing the first value and second value comprises considering at least one of the group consisting of: an expected range of values for the physiological parameter, historical information, patient information, a statistical measure, noise associated with the signal, a confidence indicator.

4. The method of claim 1 further comprising:
assigning a first weight to the first value; and
assigning a second weight to the second value;
wherein determining, based at least in part on the analysis of the first value and the second value, the desired value for the physiological parameter comprises calculating a weighted average of the first value and the second value.

5. The method of claim 4 wherein the first weight and the second weight are based on at least one of the group consisting of: an expected range of values for the physiological parameter, historical information, patient information, a statistical measure, noise associated with the signal, a confidence indicator.

6. The method of claim 1 wherein the physiological parameter comprises one of the group consisting of: blood oxygen saturation, pulse rate, respiration rate, blood pressure, and respiration effort.

7. The method of claim 1 wherein the continuous wavelet transform technique comprises:
performing a continuous wavelet transform of the at least one physiological signal;
generating at least one scalogram based at least in part on the continuous wavelet transform; and
analyzing features in the at least one scalogram.

8. The method of claim 7 wherein analyzing features in the at least one scalogram comprises a technique selected from the group consisting of: following a ridge, generating a Lissajous figure based on amplitude values of two scalograms, and determining a ratio of an amplitude value of one scalogram to an amplitude value of another scalogram.

9. The method of claim 1 wherein the non-continuous wavelet transform technique comprises a technique selected from the group consisting of: a time domain technique and a spectral technique.

10. A system for determining a physiological parameter from a physiological signal, the system comprising:
a sensor configured to generate at least one physiological signal; and
a processor configured to:
calculate a first value for a physiological parameter based at least in part on the at least one physiological signal using a continuous wavelet transform technique;
calculate a second value for the physiological parameter based at least in part on the at least one physiological signal using a non-continuous wavelet transform technique;
analyze the first value and the second value; and
determine, based at least in part on the analysis of the first value and the second value, a desired value for the physiological parameter.

11. The system of claim 10 wherein the at least one physiological signal comprises a photoplethysmogram signal.

12. The system of claim 10 wherein analyze the first value and second value comprises considering at least one of the group consisting of: an expected range of values for the physiological parameter, historical information) patient information, a statistical measure, noise associated with the signal, a confidence indicator.

13. The system of claim 10 wherein the processor is configured to:
assign a first weight to the first value; and
assign a second weight to the second value; and
wherein determine, based at least in part on the analysis of the first value and the second value, the desired value for the physiological parameter comprises calculating a weighted average of the first value and the second value.

14. The system of claim 13 wherein the first weight and the second weight are based on at least one of the group consisting of: an expected range of values for the physiological parameter, historical information, patient information, a statistical measure, noise associated with the signal, a confidence indicator.

15. The system of claim 10 wherein the physiological parameter comprises one of the group consisting of: blood oxygen saturation, pulse rate, respiration rate, blood pressure, and respiration effort.

16. The system of claim 10 wherein the continuous wavelet transform technique is performed by the processor, further configured to:
perform a continuous wavelet transform of the at least one physiological signal;
generate at least one scalogram based at least in part on the continuous wavelet transform; and
analyze features in the at least one scalogram.

17. The system of claim 16 wherein the analyze features in the at least one scalogram comprises a technique selected from the group consisting of: following a ridge, generating a Lissajous figure based on amplitude values of two scalograms, and determining a ratio of an amplitude value of one scalogram to an amplitude value of another scalogram.

18. The system of claim 10 wherein the non-continuous wavelet transform technique comprises a technique selected from the group consisting of: a time domain technique and a spectral technique.

19. A computer-readable medium for use in determining a physiological parameter from a physiological signal, the computer-readable medium having computer program instructions recorded thereon for:
receiving at least one physiological signal;
calculating a first value for a physiological parameter based at least in part on the at least one physiological signal using a continuous wavelet transform technique;
calculating a second value for the physiological parameter based at least in part on the at least one physiological signal using a non-continuous wavelet transform technique;
analyzing the first value and the second value; and
determining, based at least in part on the analysis of the first value and the second value, a desired value for the physiological parameter.
